(19) United States
(12) Patent Application Publication
(10) Pub. No.: US 2009/0216168 A1
(43) Pub. Date: Aug. 27, 2009

(54) MULTI-LAYER ABSORBENT WOUND DRESSING WITH A HYDROPHILIC WOUND CONTACT LAYER

(75) Inventor: Axel Eckstein, Heidenheim (DE)

Correspondence Address: Brinks Hofer Gilson & Lione/Ann Arbor, 524 South Main Street, Suite 200, Ann Arbor, MI, 48104, US

(73) Assignee: Paul Hartmann AG, Heidenheim (DE)

(21) Appl. No.: 12/437,266

(22) Filed: May 7, 2009

Related U.S. Application Data
(63) Continuation of application No. PCT/EP2007/009119, filed on Oct. 20, 2007.

(30) Foreign Application Priority Data
Nov. 7, 2006 (EP) 06 023 099.2

(51) Int. Cl. A61F 13/02

(52) U.S. Cl. 602/43

(57) ABSTRACT

The present invention relates to wound dressings, particularly for the treatment of medium to severely exudative wounds, and their use in modern wound treatment. As multi-layer wound dressings, these wound dressings contain a carrier layer, an absorbing layer, and a hydrophilic wound contact layer connected to the absorbing layer, wherein the wound contact layer comprises a hydrophilic polyurethane elastomer.

### CROSS-REFERENCE TO RELATED APPLICATIONS

This application is a continuation of International Application No. PCT/EP2007/009119, filed on Oct. 20, 2007, which claims the benefit of EP 06 023 099.2, filed Nov. 7, 2006. The disclosures of the above applications are incorporated herein by reference.

### FIELD

The present invention relates to wound dressings, particularly for the treatment of moderately to severely exudative wounds, and to their use in modern wound treatment.

### BACKGROUND

The statements in this section merely provide background information related to the present disclosure and may not constitute prior art.

The treatment of skin wounds is based on the capacity of the skin to regenerate epithelia as well as connective and supporting tissue. It is marked by a complex occurrence of interweaving cell activities which drive the healing process forward by steps. In the literature, regardless of the type of wound, three basic healing phases of a wound are described. These include the inflammatory or exudative phase for hemostasis and wound purification (Phase 1, purification phase), the proliferative phase to build up granulation tissue (Phase 2, granulation phase), and the differentiation phase for epithelization and scar formation (Phase 3, epithelization phase).

Numerous recommendations are described in the literature to support the individual wound healing phases. For example, the European patents EP 59048, EP 59049, EP 97517, EP 99748 and EP 106439 each describe a non-wound-adhering wound dressing which features a polyurethane foam as the absorbent layer. These wound dressings comprise an elastomer net or a perforated film of polyurethane as the wound contact layer.

EP 486522 claims a flexible wound dressing comprising a flat layer of a foam material, preferably a polyurethane foam, which in operating status lies opposite the wound and which is provided with a discontinuous adhesive layer. On the side facing away from the wound, the wound dressing features a water-impermeable but water vapor-permeable material, preferably a polyurethane film. At least two opposite sides of the wound dressing should feature flattened margins.

WO 94/07935 describes a wound dressing which comprises of a self-adhering hydrophilic polyurethane gel foam, that can be obtained from a polyhydroxy compound, a polyisocyanate, a non-aqueous foaming agent, and a water-absorbing superabsorber.

Further, WO 97/42985 describes a wound dressing which comprises an absorbent foam layer and an adhesive, hydrophobic gel that is applied to it. The foam material is porous or perforated, whereby the holes are proximal to the wound in the properly used state of the wound dressing, and the walls of the holes are coated with the gel.

Furthermore, WO 2004/060359 describes a wound dressing which comprises an absorbent foam material that is provided with openings. Superabsorbent particles are applied to the openings, whereby the openings are closed by a back layer. A hydrophobic elastomer silica gel is applied to the absorbent foam material surface which faces the wound in the properly used state.

### SUMMARY

A multi-layer wound dressing is provided which comprises a carrier layer, an absorbent layer, and a hydrophilic wound contact layer, whereby the wound contact layer is connected to the absorbent layer and a hydrophilic polyurethane elastomer. In particular, the wound contact layer comprises a hydrophilic polyurethane elastomer.

A special advantage of this wound dressing lies in the fact that a separating layer is created between the absorbent layer and a wound by means of the wound contact layer on the one hand, and thus even materials which tend towards wound adhesion can be used as the absorbent layer and, on the other hand, a non-wound-adhering wound contact layer is created using the hydrophilic polyurethane elastomer which, owing to its hydrophilia, allows improved transport of wound fluid from the wound. Furthermore, it has been shown that this wound dressing prevents or at least restricts maceration, that is, the softening and associated damage of the skin surrounding the wound. Thus, a wound dressing can be provided which protects the skin surrounding the wound and which largely promotes wound healing.

Understood hereby as the polyurethane elastomer in conjunction with the present disclosure, is an elastomer compound which can be manufactured from at least one di- or polyisocyanate (isocyanate component) and at least one diol or polyol (polyol component) by polyaddition reactions, without the hydrophilic contact layer or the elastomer being present as foam. Both prepolymer compounds and monomer compounds can be used as a suitable isocyanate component or polyol component.

Examples of suitable di- or polyisocyanates in accordance with the disclosure include MDI (diphenylmethane diisocyanate), TDI (toluene diisocyanate), XDI (xylylene diisocyanate), NDI (naphthalene diisocyanate), phenylene diisocyanate, dicyclohexylmethane diisocyanate, butane-1,4-diisocyanate, tetramethoxy butane-1,4-diisocyanate, hexane-1,6-diisocyanate, ethylene diisocyanate, 2,2,4-trimethyl hexamethylene diisocyanate, ethyl ethylene diisocyanate, dicyclohexylmethane diisocyanate, 1,4-diisocyanatocyclohexane, 1,3-diisocyanatocyclohexane, 1,2-diisocyanate, 1,3-diisocyanatocyclopentane, 1,2-diisocyanatopentane, 1,2-diisocyanatocyclobutane, 1-isocyanatomethyl-3-isocyanato-1,5,5-trimethylcyclohexane (isophorone-diisocyanate, IPDI), 1-methyl-2,4-diisocyanatocyclohexane, 1,6-diisocyanato-2,2,4-trimethylhexane, 1,6-diisocyanato-2,4,4-trimethylhexane, 5-isocyanato-1-(2-isocyanatoeth-1-yl)-1,3,3-trimethylcyclohexane, 5-isocyanato-1-(3-isocyanatoprop-1-yl)-1,3,3-trimethylcyclohexane, 5-isocyanato-1-(4-isocyanatobut-1-yl)-1,3,3-trimethylcyclohexane, 1-isocyanato-2-(3-isocyanatoprop-1-yl)-cyclohexane, 1-isocyanato-2-(2-isocyanatoeth-1-yl)-cyclohexane, 2-heptyl-3,4-bis(9-isocyanatononyl)-1-pentylcyclohexane, norbonane diisocyanatomethyl, chlorinated, bromated, sulfuric, or phosphoric aliphatic or cycloaliphatic diisocyanates, in particular dimerized or trimerized types.

According to one form of the present disclosure, polyurethane elastomers produced from aliphatic or cycloaliphatic di- or polyisocyanates are preferred.

In particular, linear aliphatic or cycloaliphatic diisocyanates are preferred, whereby five- or six-member cycloaliphatic diisocyanates are also preferred. In an especially preferred embodiment, isophorone diisocyanate is used as a six-member cycloaliphatic diisocyanate which provides very good water vapor permeability.

Additional isocyanate components are prepolymers from aliphatic or cycloaliphatic di- or polyisocyanates and di- or polyols; also preferred are prepolymers from cycloaliphatic diisocyanates, whereby polyether polyols or polyester polyols are used as the polyols in particular.

Generally the polyurethane prepolymers useable in the scope of the present disclosure feature a molecular weight of about 500 g/mol to about 15,000 g/mol, preferably about 500 g/mol to about 10,000 g/mol, and especially preferably, about 700 g/mol to about 4,500 g/mol.

Examples of suitable diols or polyols in accordance with the disclosure include oxyalkyl polymers, preferably 2, 3, 4, 5 or 6 hydroxyl groups-bearing polyether polyols with OH-numbers from 20 to 112 and an ethylene oxide content ≧10% by weight, preferably 10 to 40% by weight, especially preferably about 10 to 20% by weight; polyacryl polyols; polyester polyols; polyolefin polyols; polythiol polyols; and polyamine compounds. The glass transition temperatures here should be as low as possible, namely below about 20° C., preferably below about 0° C., but most preferred below about −10° C.

Polyether polyols with molecular weights between about 600 and about 12,000 are preferred and can be obtained in accordance with a known method, for example, by a reaction of the starter compounds with a reactive H-atom with alkylene oxides (if applicable ethylene-, and/or propylene oxide, preferably propylene oxide, butylene oxide, styrene oxide, tetrahydrofuran or epichlorhydrin or mixtures of two or more of them). Likewise, tetramethylene ether glycols can be used. Also possible are further modifications, for example, with monoethylene glycol (MEG), dipropylene glycol (DPG), trimethylol propane (TMP). Aliphatic polyether polyols are preferred today for use in medicine.

Suitable starter compounds include, for example, water, ethylene glycol, propylene glycol-1,2 or -1,3, butylene glycol-1,4 or -1,3, hexane diol-1,6, octane diol-1,8, pentane diol-1,5, heptane diol-1,7, and their higher homologues, neopentyl glycol, 1,4-hydroxymethyl cyclohexane, 2-methyl-1,3-propandiol, glycerol, trimethylol propane, 2,2-(bis-4,4′-hydroxyphenyl)-propane, trimethylolpropane, glycerol or pentaerythritol, hexanetriol-1,2,6, butanetriol-1,2,4 trimethylol ethane, mannitol, sorbitol, methyl glycosides, sugar, phenol, isononyl phenol, resorcin, hydroquinone, 1,2,2- or 1,1,2-tris-(hydroxy phenyl)-ethane, ammonia, methylamine, ethylene diamine, tetra- or hexamethylene amine, triethanolamine, aniline, phenylenediamine, 2,4- and 2,6-diaminotoluene and polyphenyl polymethylene polyamine, as they can be obtained through aniline-formaldehyde condensation or mixtures of the above starting compounds.

Equally suited as a diol- or polyol component are the polyacrylates which carry OH groups. These are obtained, for example, by polymerization of ethylenically unsaturated monomers which carry an OH group. Such monomers can be obtained, for example, by esterification of ethylenically unsaturated carboxylic acids and difunctional alcohols, whereby the alcohol generally is present with a slight excess. Such unsaturated carboxylic acids include, for example, acrylic acid, methacrylic acid, crotonic acid, or maleic acid. Esters carrying the corresponding OH-groups include, for example, 2-hydroxyethyl acrylate, 2-hydroxyethyl methacrylate, 2-hydroxy-propyl acrylate, 2-hydroxypropyl methacrylate, 3-hydroxypropyl acrylate, or 3-hydroxypropyl methacrylate or mixtures of two or more of them.

Also suited as diol or polyol components are polyester polyols, in particular with a molecular weight of about 200 to about 10,000. Thus, for example, polyester polyols can be used which occur by the reaction of low-molecular alcohols, in particular ethylene glycol, diethylene glycol, neopentyl glycol, hexanediol, butanediol, propylene glycol, glycerin or trimethylolpropane with caprolactone. Likewise suited as polyfunctional alcohols for the production of polyester polyols are 1,4-hydroxy-methyl cyclohexane, 2-methyl-1,3-propanediol, butanetriol-1,2,4, triethylene glycol, tetraethylene glycol, polyethylene glycol, dipropylene glycol, polypropylene glycol, dibutylene glycol and polybutylene glycol. Additional suitable polyester polyols may be produced by polycondensation. Thus, difunctional and trifunctional alcohols with an excess of dicarboxylic acids, and or tricarboxylic acids, or their reactive derivatives are condensed to polyester polyols. Suitable dicarboxylic acids include, for example, adipinic acid or succinic acid and its higher homologues with up to 16 C-atoms, also unsaturated dicarboxylic acids such as maleic acid or furmaric acid, as well as aromatic dicarboxylic acids, in particular the isomers of phthalic acid, such as phthalic acid, isophthalic acid, or terephthalic acid. Citric acid or trimellitic acid is suitable as tricarboxylic acids. The named acids can be used individually or as a mixture of two or more. Especially suited are polyester polyols of at least one of the named dicarboxylic acids and glycerol which feature a residual content of OH-groups. Especially suited alcohols include hexanediol, ethylene glycol, diethylene glycol or neopentyl glycol, or mixtures of two or more of them. Especially suited acids include isophthalic acid or adipinic acid or their mixtures. Polyester polyols with high molecular weight in particular in the region from >5000 g/mol include, for example, the reaction products of polyfunctional, preferably difunctional alcohols (possibly with small quantities of trifunctional alcohols) and polyfunctional, preferably difunctional carboxylic acids. In place of free polycarboxylic acids, the corresponding polycarboxylic acid anhydrides or corresponding polycarboxylic acid esters with alcohols with preferably 1 to 3 C-atoms can be used (when possible). The polycarboxylic acids can be aliphatic, cycloaliphatic, aromatic, or heterocyclic polycarboxylic acids. They can, if necessary, be substituted, if applicable by alkyl groups, alkenyl groups, ether groups, or halogens. The polycarboxylic acids include, for example, succinic acid, adipinic acid, suberic acid, azelaic acid, sebacic acid, phthalic acid, isophthalic acid, terephthalic acid, trimellitic acid, phthalic acid anhydride, tetrahydrophthalic acid anhydride, hexahydrophthalic acid anhydride, tetrachlorophthalic acid anhydride, endomethylene tetrahydrophthalic acid anhydride, glutaric acid anhydride, maleic acid, maleic acid anhydride, fumaric acid, dimer fatty acid or trimer fatty acid or mixtures of two or more of them. If necessary, subordinate quantities of monofunctional fatty acids can be present in the reaction mixture. The polyesters can, for example, feature a small portion of carboxylene groups. Polyesters obtainable, for example, from lactones, for example epsilon-caprolactone or hydroxycarboxylic acids, for example omega-hydroxycapronic acid, can be used.

In addition, the previously named diols or polyols can be mixed. Compatibility must thereby be considered. Aliphatic polyester polyols are utilized in connection with the present disclosure.

If necessary, the inventive polyurethane elastomer can also contain additives, such as, for example, softeners, stabilizers such as anti-oxidants or photo-stabilizers, tackifiers, dyes, fillers, thickeners, and rheology additives.

The softener can be, for example, phthalic acid derivatives, for example, which feature 6 to 12 carbon atoms and are estered with a linear alcohol, e.g. dioctyl phthalate. Polyethylene glycols and derivatives, vegetable and animal oils, such as glycerol esters of fatty acids and their polymerization products and benzoate compounds (benzoate softeners, for example sucrose benzoate, diethylene glycol dibenzoate and/or diethylene glycol benzoate in which about 50 to about 95% of all hydroxyl groups are estered. Phosphate-softeners, for example, t-butylphenyl diphenyl phosphate, polyethylene glycol and its derivatives, for example diphenyl ether of poly(ethylene glycol), liquid resin derivatives, for example methyl ester of hydrated resin, are likewise suited as softeners. Especially preferred are the aliphatic diesters, such as adipinic- or sebacic dinonyl ester.

Stabilizers (anti-oxidants) utilized in the scope of the present disclosure include phenols, such as BHT, Irganox® 1010, 1076, 1330, 1520 (Ciba Specialty Chemicals) as well as tocopherols. In one form, a vitamin E (alpha-tocopherol) is used. Likewise, polyfunctional phenols can be used, as well as sulfur and phosphorous compounds and/or 1,3,5-trimethyl-2,4,6-tris(3,5-di-tert-butyl-4-hydro-xybenzyl)benzene; pentaerythritol tetrakis-3-(3,5-di-tert-butyl-4-hydroxyphenyl)propionate; n-octadecyl-3,5-di-tert-butyl-4-hydroxyphenyl)propionate; 4,4-methylene bis(2,6-di-tert-butyl-phenol); 4,4-thiobis(6-tert-butyl-o-cresol); 2,6-di-tert-butyl phenol; 6-(4-hydroxyphenoxy)-2,4-bis(n-octylthio)-1,3,5-triazine; di-n-octadecyl-3,5-di-tert-butyl-4-hydroxybenzylphosphonate; 2-(n-octylthio)ethyl-3,5-di-tert-butyl-4-hydroxy-benzoate; and sorbithexa[3-(3,5-di-tertbutyl-4-hydroxy-phenyl)-propionate]. Suited as photo stabilizers are, for example, Tinuvin®-products (Ciba Specialty Chemicals), benzotriazole compounds, salicylates, substituted tolyl- and metal-chelate compounds, and benzotriazoles derivatives. Combinations of the above compounds are also possible. The usual amounts are between about 0.1 and 10% by weight.

Other additives can be used to control specific properties of the polyurethane elastomer according to the state of the art. These include, for example, dyes, such as titanium dioxide, fillers such as talcum, chalk, clay and the like. It is likewise possible to include certain hydrophilic polymers, for example, PVOH (polyvinyl alcohol), polyvinyl pyrrolidone, hydroxypropyl cellulose, polyvinyl methyl ether and cellulose esters, especially their acetates with a log degree of substitution. These can enhance the wettability of the polyurethane elastomer. The fillers are understood to be the fillers usually used in polyurethane chemistry. These also include zinc oxide, titanium oxide, and silicic acid derivatives (e.g. Aerosile® (Degussa)). Other additives to be mentioned include, for example, the short fibers on an organic or inorganic basis, (e.g. glass fibers, textile fibers).

In order be able to improve the wetting of the substrate, the usual wetting agent can be added to the polyurethane elastomer: for example poloxamers (copolymer of polyoxyethylene and polyoxypropylene), sorbitan esters, fatty acids such as Span® (Sigma-Aldrich), esters of polyoxyethylene sorbitan and fatty acids such as Polysorbate or Polysorbate® (Spectrum Chemical), polyoxyethylated hydrated castor oils, such as for instance Cremophor® (BASF), polyoxyethylene stearate, e.g. Myrj® (Uniqema) or each combination of these wetting agents. Preferably a polysorbate should be used as a wetting agent.

In addition, the polyurethane elastomer can contain tackifier resins. These can be natural, modified natural, and synthetic resins, typically with a molecular weight of up to about 1500 g/mol. The compatibility of the resins with the other components is in each case to be tested in routine tests of the prior art. For example, hydrocarbon resins are suitable, in particular C5- to C9-resins, preferably C9-resins modified with C5-resins, and the like. All the hydrocarbon resins can be partially or fully hydrated. Equally usable are natural resins, such as balsamic resin or tall resin. The indicated resins can also be estered with the corresponding polyfunctional alcohols, such as pentaerythritol ester, glycerol ester, diethylene glycol ester, triethylene glycol ester or methyl ester and thus utilized. Known commercial products include, e.g., “Staybelite” ster 10, “Foral” 85-105, “Hercolyn” D, “Alresen” 214 R, “Alresen” 191 R, “Alresen” 500 R 80 and “Cellolyn” 21 s. Polyterpene resins, such as the terpene phenol resins can likewise be formulated as tackifier resins, as well as the synthetic resins: ketone-, coumarone- and indene resins, and also hydrocarbon resins are also possible, for example, under such trade names as “Ketonharz” N, “Lutonal” J 30, “Lutonal” J 60, “Vinnapas” B 17, “Vinnapas” 50 V 1, Kohlenwasserstoffharz 95 KW 10, KW 20 and KW 30. Polyvinyl ether is also an effective tackifier. Acrylate resins can likewise be used alone or in mixtures with the above-named tackifiers.

Thus, according to another form of the present disclosure, a wound dressing contains a polyurethane elastomer as a wound contact layer that can be obtained by a polymerization of at least one aliphatic and/or cycloaliphatic isocyanate component with a polyether polyol component.

In particular, the wound contact layer contains a polyurethane elastomer, especially an adhesive polyurethane elastomer or a polyurethane elastomer adhesive which is obtainable by polymerization of isophorone diisocyanate or a modified isophorone diisocyanate with at least one diol- or polyol component, preferably 2, 3, 4, 5 or 6 hydroxyl groups featuring polyether polyols with OH-numbers from about 20 to about 112 and an ethylene oxide (EO)-content of ≧10% by weight, preferably about 10 to 40% by weight, but most preferably about 10 to 20% by weight.

These wound contact layers feature an especially good water vapor permeability. In particular thereby is a wound dressing comprising a hydrophilic wound contact layer, whereby the wound contact layer comprising a hydrophilic polyurethane elastomer which features a water vapor permeability of >2,000 g/m2/24 h, in particular of >2,500 g/m2/24 h with a full, uninterrupted surface area of 100 g/m2 on a carrier material is also within the scope of the present disclosure, whereby the water vapor permeability is measured in accordance with DIN EN 13726-2: 2002-MVTR with contact to water (cf. Test 4b).

The hydrophilic polyurethane elastomer also preferably involves a water-free elastomer. Understood hereby in connection with the present disclosure as a water-free polyurethane elastomer or that water-free compound or components is an elastomer, a compound or component which contains less than about 4% by weight water based on the weight of the particular elastomer, compound, or component. In particular, such an elastomer, compound, or component contains less than about 2% by weight water, and especially less than about 1% by weight water, and most preferably less than about 0.5% by weight water.

These hydrophilic polyurethane elastomers can further feature adhesive properties. In accordance with another form of the present disclosure, the wound contact layer therefore also comprises an adhesive hydrophilic polyurethane elastomer or a hydrophilic polyurethane elastomer adhesive. The adhesive polyurethane elastomer or the polyurethane elastomer adhesive thereby is an elastomer which features a weak to medium adhesiveness on human skin or tissue. In particular, a wound dressing in accordance with the disclosure features an adhesive strength of about 0.02 to 5 N/25 mm, in particular 0.02 bis 3 N/25 mm and quite especially preferably 0.02 to 2 N/25 mm. Here the adhesive strength is measured in accordance with Test 1 (cf. Test Methods) against steel with a traction angle of 90°.

In particular the hydrophilic polyurethane elastomer involves a hydrophilic polyurethane adhesive which features less than about 4% by weight, in particular less than about 3% by weight water, but most preferably less than about 1.5% by weight water. In another form, the hydrophilic polyurethane elastomer is a water-free polyurethane adhesive.

In accordance with the present disclosure, the wound contact layer is to be understood as a layer which features a first and a second side, whereby the first side forms a direct contact with a wound in properly used status of the wound dressing. With regard to the form and extent of the wound contact layer, no limits are set here. This wound contact layer can be fully contiguous with respect to the absorbent layer and or feature a uniform or profiled layer thickness and/or feature a regular or an irregular pattern.

In accordance with the present disclosure, the wound contact layer must be connected to the absorbent layer. This is understood to mean that the wound contact layer in at least one region of its second side is in direct contact with a first side of the absorbent layer, which in properly used status is facing the wound. In a special preference, the wound dressing comprises a wound contact layer whose second side is fully contiguous with the first side of the absorbent layer. Alternatively, the wound contact layer can also be connected integrally to the absorbent layer. This is understood to mean that the two mutually connected and abutting layers form a transitional layer at the boundary surfaces which cannot be separated. A laminate is furthermore provided by means of these integrally connected layers which does not comprise separable layers that are chemically and/or physically connected to one another.

Accordingly, a multi-layer wound dressing, comprising a carrier layer, an absorbent layer with a first and a second side, and a hydrophilic wound contact layer with a first and second side, whereby the second side of the wound contact layer is connected completely contiguous to the first side of the absorbent layer and comprises a hydrophilic polyurethane elastomer, is likewise an object of the polyurethane elastomer. In a special preference, this wound dressing also features a wound contact layer which, with respect to the first side of the absorbent layer, is fully contiguous. This wound dressing features a wound contact layer which is integrally connected to the absorbent layer and which in particular is fully contiguous with respect to the first side of the absorbent layer.

Finally, it can also be provided that the wound dressing features a wound contact layer which, with respect to the first side of the absorbent layer, is not fully contiguous, but leaves open individual regions of the absorbent layer, for example, in order to apply an adhesive here to secure the wound dressing to the skin of a patient. Alternatively, the wound dressing also need not be configured to be fully contiguous with respect to the absorbent layer, but rather a discontinuous wound contact layer is chosen which provides regularly or irregularly arranged openings. These openings provide improved passage for wound fluids from the wound to the absorbent layer.

Further, wound dressings have been shown to be especially advantageous embodiments when they feature a wound contact layer whose layer thickness is less than 1000 μm. A wound dressing in accordance with the present disclosure features a wound contact layer with a layer thickness of 10 to 1000 μm, quite preferably 10 to 500 μm, and most preferably 10 to 250 μm. Wound dressings with such layer thicknesses, on the one hand, do not demonstrate wound adhesion and, on the other, they have the capacity to absorb a wound exudate given off by a wound and to transmit it to the absorbent layer. These layer thicknesses can be the same at every point of the wound contact layer or they can assume different values in different regions of the wound contact layer.

The expression “hydrophilic,” as it is used in connection with the present disclosure, describes elastomers or the surfaces of layers which are wettable by the aqueous fluids deposited on these surfaces or elastomers (for example, aqueous bodily fluids such as wound secretions). Hydrophilia and wettability can be defined by means of a contact angle and the surface tension of the involved fluids and solids. Accordingly, in connection with the polyurethane, a hydrophilic layer or a hydrophilic polyurethane elastomer should be understood to mean a layer or an elastomer which features a contact angle less than 90° with respect to the water or when the water tends to spread spontaneously on the surface of the layer or elastomer, whereby the definition of the contact angle is made analogously to DIN EN 828. Both processes are generally coexistent. Conversely, a layer or an elastomer is termed hydrophobic, when a contact angle of greater than or equal to about 90° is formed and no spread is observed.

Accordingly, the wound dressing of the disclosure includes a hydrophilic polyurethane elastomer or a hydrophilic wound contact layer which features a contact angle with respect to water of less than about 90°, especially less than about 75°, and further less than about 65°, whereby the contact layer is obtained analogously to DIN EN 828.

Further, in accordance with the present disclosure, polyurethane elastomers may be used, which themselves feature absorbent properties. Some polyurethane elastomers can absorb at least 50% of their own weight in aqueous fluids. Thus, a multi-layer wound dressing comprising a carrier layer, an absorbent layer, and a hydrophilic wound contact layer is likewise described herein, whereby the wound contact layer is connected to the absorbent layer and comprises a hydrophilic polyurethane elastomer which absorbs at least about 50% by weight of salt solution, based on its own weight. Such a polyurethane elastomer may absorb about 50 to about 200% by weight, or even about 50 to about 150% by weight of saline solution based on its own weight, whereby a determination of the absorption performance is carried out analogously to DIN-EN 13726-1 (2002).

The absorbent layer can be any of the materials usually utilized in modern wound treatment. To be mentioned herein are those materials which can be utilized in wet wound therapy. Those absorbent materials which both pick up wound secretions, and thus act as absorbing agents, and also give off moisture to the wound are disclosed herein. Also, those absorbent layers which are transparent or translucent may be used. In accordance with the polyurethane elastomer described herein, the wound dressing comprises as its absorbent layer a hydrophilic polymer foam, an absorbent liner or nonwoven, a polymer matrix comprising at least one hydrocolloid, a freeze-dried foam, or a combination thereof.

If a hydrophilic polymer foam is used as the absorbent layer, it has been shown that the wound dressing adapts well to the wound to be treated. Hydrophilic polyurethane foams are especially suited as polymer foams. Accordingly, the present wound dressing comprises an absorbent layer made from a hydrophilic polyurethane foam. These polyurethane foams feature a free absorption of at least about 10 g/g, or at least about 12 g/g, and or even about 15 g/g, whereby the free absorption is determined in accordance with DIN-EN 13726-1 (2002). Another feature is that these foams feature a pore size averaging less than about 1000 μm, in particular about 200 to about 1000 μm, and even about 200 to about 700 μm. It can be provided hereby that the pore size on a first surface of the absorbent layer differs from the pore size of a second surface of the absorbent layer. An additional preference entails the hydrophilic polyurethane foams featuring a density of less than about 150 kg/m3, or less than about 140 kg/m3, or evenless than about 70 to 120 kg/m3.

In a variation, an absorbent layer features water insoluble fibers of cellulose, especially largely delignified technical cellulose fibers, in particular wood pulp fibers, especially with a fiber length of <about 5 mm. The fiber material also may contain hydrophilic fiber material from regenerated cellulose, carboxymethyl cellulose, carboxyethyl cellulose, hydroxymethyl cellulose or hydroxyethyl cellulose. In addition, a fiber mixture can be provided, of cellulose, regenerated cellulose, carboxymethyl cellulose, carboxyethyl cellulose, hydroxymethyl cellulose or hydroxyethyl cellulose fibers and fibers of polyethylene, polypropylene, or polyester. In addition, the absorbent layer comprises a mixture of cellulose fibers, polypropylene fibers, and particle-shaped superabsorbent polymers, preferably of cross-linked sodium polyacrylate.

Another alternative wound dressing comprises an absorbent layer that comprises of a hydrophobic matrix into which hydrocolloids have been dispersed. In accordance with the present disclosure, a hydrocolloid should be understood as a material which is a hydrophilic, synthetic, or natural polymer material, that is soluble or absorbent and/or swelling in water. An absorbent layer comprises a hydrocolloid made of a synthetic or natural polymer material which is selected from the group alginic acid and its salts, as well as its derivatives, chitin or its derivatives, chitosan or its derivatives, pectin, cellulose or its derivatives such as cellulose ether or cellulose ester, cross-linked or non-cross-linked carboxyalkyl cellulose or hydroxyalkyl cellulose, polyvinyl alcohol, polyvinyl pyrrolidone, agar, guargum or gelatin.

The hydrocolloid can hereby be both in the form of fibers and in the form of particles and/or fibers inside the matrix. In particular, the hydrocolloid can be in the form of particles in an adhesive polymer matrix. The adhesive polymer matrix here comprises at least one non-hydrated, partially hydrated or fully hydrated co-blockpolymer selected from the group of AB-diblock-copolymers and/or ABA-triblock-copolymers which is structured from the monomers styrene, butadiene and/or isoprene. The portion of hydrocolloid particles in the wound contact layer can be about 10 to 70% by weight based on the total weight of the absorbent layer. Such a composition is known for example from EP 1 007 597 B1.

Various materials can be used as the carrier layer. Usually textile carrier materials, nonwovens, polymer films, and polymer films are used in wound dressings. Polymer films or polymer foams can be used as a carrier layer of a wound dressing. Polymer films which feature a high water vapor permeability can also be used. Films which are manufactured from polyurethane, polyether urethane, polyester urethane, polyether-polyamide-copolymers, polyacrylate, or polymethacrylate are also suitable. Polyurethane film, polyester urethane film, or polyether urethane film are also suitable. Polymer films which feature a thickness of about 15 to about 50 μm, or about 20 to about 40 μm or even about 25 to about 30 μm. The water vapor permeability of the polymer film of the wound dressing is at least about 750 g/m²/24 h, or at least about 1000 g/m²/24 h, or even at least about 2000 g/m²/24 h (measured per DIN EN 13726). In one form, these films feature a moisture-proof adhesive marginal section. This marginal section ensures that the wound dressing is applied to its appropriate place and can be secured there. Furthermore, it is assured that no fluid can escape between the foil and the skin surrounding the surface to be treated. Those adhesives which feature in a thin application of about 20 to about 35 g/m² together with the film a water vapor permeability of at least 800 g/m²/24 h or at least about 1000 g/m²/24 h (measured per DIN EN 13726) are suitable.

Alternatively, the carrier layer can be made from a polymer foam. The polymer foam may be a polyurethane foam. These polyurethane foams are basically made from closed-pore polyurethane foams and in particular feature a thickness of about 0.01 to about 2 mm. Here basically closed-pore polyurethane foams prove to be useful, since these foams are both water-vapor permeable and also pose a barrier to dirt and bacteria. This carrier material preferably features a water vapor permeability of at least about 750 g/m²/24 h, or at least about 1000 g/m²/24 h, or even at least about 1200 g/m²/24 h (measured per DIN EN 13726).

The present wound dressing may feature, as the carrier layer and as the absorbent layer, a laminate made from two different polyurethane foams. In particular, this wound dressing comprises of a laminate from two different polyurethane foams which is laminated with a hydrophilic wound contact layer comprising an adhesive hydrophilic polyurethane elastomer on the side facing the wound when the wound dressing is improperly applied. A wound dressing comprising a carrier layer made from a first hydrophilic water vapor permeable polyurethane foam, an absorbent layer made from a hydrophilic absorbent polyurethane foam, and a hydrophilic wound contact layer connected to the absorbent layer and integrally connected thereto, which comprises an adhesive, hydrophilic polyurethane elastomer different from the hydrophilic foam, is also disclosed herein. This wound dressing also comprises an absorbent layer which features a higher free absorption than the wound contact layer.

This wound dressing is advantageously utilized as a supporting measure in modern wound treatment, since no wound adhesion occurs owing to the wound contact layer, no maceration of the skin surrounding the wound occurs, the acceptance of the wound fluids given off by the wound occurs rapidly, and the wound dressing can be secured to the skin surrounding the wound.

Furthermore, it has turned out that a wound dressing in accordance with this disclosure, can also be used for a period of up to seven days depending on the type of wound. This is possible because the wound dressing is especially effective with regard to absorption capacity. A wound dressing in accordance with the present disclosure features a maximal fluid uptake capacity (max. FL) of at least about 5,000 g/m²/24 h, or about 5,000 to about 10,000 g/m²/24 h, whereby the maximal fluid uptake capacity is determined by the following formula: max. FL=(surface weight×fluid uptake)+MVTR [g/m²/24 h), wherein the following have the meanings given.

Surface weight is the surface weight of the sample in g/m². Fluid uptake is the fluid uptake according to Test 3 (cf. test methods). MVTR is the water vapor permeability per Test 4 (cf. test methods).

In an alternative form of a wound dressing according to the present disclosure, a wound contact layer further comprises at least one means which actively supports wound healing, in particular an antimicrobial means, a vitamin or provitamin, a fatty acid or fatty acid ester, or a means which actively promotes tissue buildup.

Alternatively, the wound contact layer further comprises at least one antimicrobial means. Suited for this are antimicrobial metals or their salts, especially silver or its salts. The wound contact layer comprises an antimicrobial means and a carrier material for the antimicrobial means. A nonwoven or a textile material such as knitted fabric, warp-knitted fabric, or woven material can be used which is layered with an antimicrobial metal, for example, silver of silver salts. It is advantageous if the hydrophilic polyurethane elastomer is water-free.

Further provided is a multi-layer wound dressing comprising a carrier layer, an absorbent layer, a hydrophilic wound contact layer, and a distribution layer, whereby the wound contact layer comprises a hydrophilic polyurethane elastomer, is likewise the object of the polyurethane elastomer. In particular, the absorbent layer is connected to the wound contact layer. Such a wound dressing features an especially advantageous distribution layer between the carrier layer and the absorbent layer which in particular comprises of a hydrophilic polyurethane foam. The distribution layer ensures, that the absorbed wound fluids are distributed over the entire surface of the wound dressing, in particular over the absorbent layer, that is, that the uptake of the wound fluids is not only in the z-direction (away from the wound and towards the carrier layer), but also in the x-y direction (over the surface of the wound dressing).

The wound dressing also may comprise at least one distribution layer, Vn, between the absorbent layer and the carrier layer, whereby n=1 to 4 and Vn with n=1 is the layer closest to the skin or wound, and Vn with n=4 is the layer furthest removed from the skin or wound. This assures an especially uniform distribution of the absorbed fluids inside the wound dressing and spaced from the wound above the skin or the wound. In particular, it has turned out that a distribution layer Vn with n=1 configured from a tissue, nonwoven, woven, knitted material and/or warp knitted material is especially effective.

In addition to the distribution layer Vn, the wound dressing can comprise at least one further absorbent layer, Am, between a first distribution layer, V1, and the carrier layer, whereby m=1 to 3 and Am with m=1 is the layer closest to the skin or the wound. Here, the wound dressing which features the distribution layer can hereby exhibit a nonwoven from a hydrophilic fiber material and a gel-forming superabsorbent material. Used here as the hydrophilic fiber material, in particular, are water insoluble fibers of cellulose, in particular largely delignified technical cellulose fibers, in particular wood pulp fibers, in particular with a fiber length of <5 mm can be used. The fiber material can also contain hydrophilic fiber material from regenerated cellulose, carboxymethyl cellulose, carboxyethyl cellulose, hydroxymethyl cellulose or hydroxyethyl cellulose.

The use of a hydrophilic wound contact layer comprising a hydrophilic polyurethane elastomer for the manufacture of a means for active and passive support of tissue buildup in wounds, especially in chronic wounds, is likewise provided herein A composite of an absorbent layer directly connected to this hydrophilic wound contact layer can be used. Accordingly, further provided is a method of treating a chronic wound comprising contacting the wound with a hydrophilic wound contact layer comprising a hydrophilic polyurethane elastomer. Also provided is the method wherein treating further comprises actively or passively supporting tissue buildup in the wound.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

In the following, the principles of the present disclosure will be explained with reference to the drawings, without these drawings exerting a restrictive action regarding the invention. Alternate forms of a wound dressing of the present disclosure are shown in cross section in FIGS. 1-6.

FIG. 1 is the simplest design of a wound dressing (10) in accordance with the present disclosure. The wound dressing comprises of a wound contact layer (11) made of hydrophilic polyurethane elastomer which is applied to an absorbent polyurethane foam as an absorbent layer (12) and is integrally connected to same. As the carrier layer (13), this wound dressing features a hydrophilic, water vapor permeable, polyurethane foam. This carrier layer is impermeable to water and bacteria and features closed pores. In this wound dressing, the wound contact layer serves as a non-wound-adhering separating layer which, owing to its hydrophilia, assures rapid uptake of the wound fluid.

FIG. 2 shows a wound dressing (20) in accordance with the present disclosure as a so-called island dressing. The wound dressing comprises of an absorbent, hydrophilic polyurethane foam as an absorbent covering (22) which is fully laminated contiguously on the side turned towards a wound in the properly used status with a hydrophilic wound contact layer made from an adhesive hydrophilic polyurethane elastomer (21). On the side of the absorbent covering turned towards the wound in the properly used state, a carrier layer (23) made from a closed-cell polyurethane foam is applied by means of a acrylate adhesive (24) applied on the entire area. Both the wound contact layer (21) and the adhesive market surrounding the wound contact layer on all sides are covered with a siliconized release paper (25) before using the wound dressing. This wound dressing features two differently adhering adhesion zones, whereby the wound contact layer features an adhesive strength of about 0.10 N/25 mm (measured against steel, cf. Test 1).

FIG. 3 likewise shows a wound dressing (30) in accordance with the present disclosure as an island dressing. The wound dressing comprises of a hydrophilic wound contact layer made from a hydrophilic polyurethane elastomer (31) which is applied on the whole area of a hydrophilic polyurethane foam (32), and is integrally connected to the foam. The absorbent layer features a layer thickness of 5 mm, whereby the polyurethane foam features a pore size of about 300 to about 900 μm. On the surface of the absorbent layer opposite the wound contact layer between the carrier layer (33) and the absorbent layer (32), there is mounted a further absorbent covering (36). This absorbent cover (36) serves as a distribution layer for fluid quantities already absorbed through the wound contact layer (31) and the absorbent layer (32). The absorbent distribution layer provides uniform diisocyanate of the absorbed fluids in the x-y direction, while the absorbent layer (32), as well as the wound contact layer (31), assure absorption of the wound fluids in the z-direction, that is, at right angles to the wound surface. The carrier layer comprises of a thin polyurethane film very permeable to water vapor, featuring a layer thickness of about 70 μm. The distribution layer comprises of a distribution pad made from cellulose fibers which is secured by means of strip-applied acrylate adhesive (34) to the carrier layer (33). Hence an improved water vapor exchange can occur, in comparison with the fully coated film through areas (38) which remain free of the acrylate adhesive. Both the wound contact layer (31) and the adhesive margin (37a, 37b) made from acrylate adhesive which surrounds the wound contact layer on all sides are covered before use of the wound dressing by a siliconized release paper (35).

FIG. 4 shows an alternate form of a wound dressing (40) in accordance with the present disclosure. In this form, an absorbent pad (42) made from a hydrophilic polyurethane foam is surrounded on all sides by an adhesive hydrophilic polyurethane elastomer (41). The elastomer thus forms the wound contact layer as well as a connection between the carrier layer (43) and the absorbent layer (42). The carrier layer comprises of a closed-cell polyurethane foam which is permeable to water vapor. Both the hydrophilic polyurethane elastomer (41) and the margin (47a, 47b), which is made from an acrylate adhesive surrounding the wound contact layer on all sides, are covered by a siliconized release paper (45) before use of the wound dressing.

FIG. 5 shows a wound dressing (50) in accordance with the disclosure which features a directly connected laminate made from a carrier layer (53) and an absorbent pad (52) made from two different polyurethane foams. The carrier layer comprises of a close-celled, water vapor permeable polyurethane foam which is laminated at the margins (57a, 57b) with an acrylate adhesive, while the absorbent layer comprises of a hydrophilic polyurethane foam. In the absorbent pad strip-like openings are provided on the surface of the absorbent pad (52) facing the wound in operational status, which are filled in by a hydrophilic polyurethane elastomer (51). Alternatively (not shown here), it can also be provided that the hydrophilic polyurethane elastomer is applied in patterns on a flat surface of an absorbent pad (52). In any case, the wound dressing (50) thus features a discontinuous wound contact layer applied in patterns. For protective storage, the surface of the wound dressing facing the wound or the skin surrounding the wound is covered before its use by a polyethylene release foil (55).

FIG. 6 shows an alternative wound dressing (60) according to the present disclosure as an island dressing. This wound dressing comprises of an absorbent hydrophilic polyurethane foam layer (62) to which a wound contact layer (61) made from an adhesive, hydrophilic polyurethane elastomer is applied. A carrier material (68) layered with silver is embedded in the wound contact layer. The hydrophilic polyurethane elastomer fully encloses the carrier material configured as a net which for its part is laminated on all sides with silver. Since the hydrophilic polyurethane elastomer itself features absorbent properties owing to the fluid absorbed from the wound contact layer, the silver material can be detached from the composite, and thus an antimicrobial wound contact layer can be provided. Furthermore, thanks to the adhesive properties of the polyurethane elastomer, a secure connection can be made between the absorbent layer and the net. As protection against contamination and bacterial penetration from outside, the wound dressing also features a carrier layer (63) made from a hydrophobic polyurethane foam. In the margin region of the carrier layer which surrounds the entire wound contact layer an acrylate adhesive (67a, 67b) is applied to secure to the skin of the patient. For protected storage, the wound dressing is inserted in a sterile package (not shown here) and the entire side of the wound dressing facing the wound, when properly used, is covered with a polyethylene release foil (65) before use.

Here it should be emphasized that the characteristics listed here for the various forms of the present disclosure should are not restricted to the individual alternatives. In conjunction with the present disclosure, it is rather the case that a combination of forms or a combination of each individual characteristic of an alternative form with the characteristics of another alternative embodiment likewise may be viewed as falling within the scope of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

### Exemplary Embodiments

### A) Test Methods

1) Adhesive strength on steel (900 traction angle) according to AFERA 5001)—Test 1. The specimen to be tested is stored for 24 hours before the test in a normal climate (23° C., 50% relative humidity), after which 3 samples each 25 mm wide and 100 mm long are taken. The test piece is carefully applied by hand, and without stretching, to the steel plates (per DIN EN 1939) during which process air bubbles are avoided. A commercial reinforcement adhesive tape which is not stretchable (e.g. Tesa 4104) is applied to the non-adhesive upper side of the specimen, in order to eliminate stretching of the foam. The test piece is rolled using tape applicator D 427/1 of the Sondes Place Research Institute Co., Surrey England with 20 N/cm, (newton/cm), in a defined manner. The steel plate prepared in this way is placed in the 90° traction device of the Z-005 traction and extension machine of the Zwick-Roell Co., Ulm, Germany, and the free end of the reinforcement tape, extending over the specimen, is inserted in the clamp. At a constant traction rate of 300 mm/min, the force progression required to separate the sample from the steel plate is measured. The adhesive strength is determined using a suitable PC program in accordance with DIN 53 539 (Method C).

2) Adhesive strength on steel (180° traction angle) according to AFERA 5001—Test 2. The specimen to be tested is kept for 24 hours before the test in a normal climate (23° C., 50% relative humidity) after which 3 samples each 25 mm wide and 100 mm long are removed. The test piece is carefully placed by hand with stretching on scratched steel plates (from the Scapa Medical Co., Bedfordshire, UK) during which process air bubbles are avoided. On the non-adhesive upper side of the specimen, a commercial reinforcement tape which is not stretchable (e.g. Tesa 4104) is placed, in order to eliminate the stretching of the foam. The test piece is manually rolled with the help of a roller with a of weight 3 kg at a rate of around 1 cm/sec manually. The steel plate prepared in this way is placed in the Z-00590° traction device of the traction and extension machine of the Zwick-Roell Co., Ulm, Germany, and the free end of the reinforcement tape which extends over the specimen is inserted in the upper clamp. At a constant traction rate of 300 mm/min, the force progression required to separate the sample from the steel plate is measured. The adhesive strength is determined using a suitable PC program in accordance with DIN ISO 6133 (Method C).

3) Free absorption of saline solution—Test 3. The free absorption of physiological sodium chloride solution (0.9% by weight, NaCl in water) is determined in accordance with DIN EN 13726-1 (2002). The only difference from DIN EN 13726-1 (2002) is the use of NaCl solution in place of NaCl and CaCl2 solution, whereby the following applies:

Fluid uptake (1)=(M2−M1)/M1[g/g],

where M2=the wet weight of the sample in g

M1=the dry weight of the sample in g

4a) Water vapor penetration (MVTR, on contact with water vapor)—Test 4a.

The water vapor penetration is determined in accordance with DIN 13726-2 (2002). A circular sample is cut from the wound dressing in such a way that it can be securely clamped over the opening of the testing device, so that no fluid can leak out. The sample is secured to the testing device in such a way, that the wound contact layer has direct contact with the forming water vapor. Prior to this, the cylinder was filled with 20 ml of distilled water. The cylinder is then exposed for 24 h to a controlled climate of 37° C. and a relative air humidity of <20%. After the test period, the sample is weighed and the following calculation made:

MVTR=(M1−M2·10000)/A [g/m²24 h] where:

MVTR is the moisture vapor transition rate=water vapor permeability,

M1 is the initial sample weight in grams,

M2 is the final sample weight in grams, and

A=the surface of the cylindrical hole of the testing device in cm².

4b) Water vapor penetration (MVTR on contact with water)—Test 4b. The measurement is in accordance with measurement 4a, with the only difference that the test device is turned around, so that the wound contact layer is in direct contact with the water column.

5) Determination of maximal fluid uptake capacity—Test 5. The maximal fluid uptake capacity (max. FL) is a composite value which is determined from the water vapor permeability in accordance with Test 4a, the surface weight of the sample in g/m2, as well as the fluid uptake in accordance with Test 3. Here, the free absorption in accordance with Test 3 is equated with the free absorption in 24 h. The value indicates the absorption performance of the wound dressing allowing for the actually utilized materials and dimensions. The maximal fluid uptake capacity is calculated by:

max.FI.=(surface weight×fluid uptake)+MVTR[g/m²24 h] (3)

Contact angle measurement T—Test 6.

The contact angle measurements were made by the East Thuringian Materials Testing Company for Textiles and Plastics mbH, Rudolstadt, Germany. The measurement of the contact angle is made using the DSA 100 testing device of the Kruss Co., Hamburg, Germany in accordance with DIN EN 828, whereby demineralized water serves as the test fluid. For testing of the contact angle, the elastomer is fully applied to a commercial polyurethane film (VP-940-2, Collano Co., Buxtehude, Germany). Demineralized water is applied to the elastomer.

7) Measurement of separating force of the wound dressing from a glass/fibrin wound model—Test 7. The test is used to assess how readily a wound dressing can be separated from an in-vitro fibrin layer. The following reagents are required:

i) PBS (phosphate buffered saline) buffer, pH 7.4, Sigma-Aldrich Co., Steinheim Germany, Art. No. P-5368. The content of a pack is dissolved with demineralized water to 1000 ml;

ii) Thrombin from beef plasma, 50 NIH-U/mg, Merck Co., Darmstadt, Germany, item No. 112374. Ten mg of thrombin are weighed in a PP centrifuge tube and dissolved with 10 ml of PBS buffer solution which is kept at 37° C. in a vortex shaker. One part of the thrombin solution is diluted with 9 parts of PBS buffer solution (=5 NIH-U/ml). The mixture must be restarted for each test!

iii) 250 mg of fibrinogen (from human plasma (95% clottable protein), Sigma-Aldrich Co., Item No. F-4883, are weighed in a large centrifuge tube and dissolved with 100 ml of BPS buffer solution which is kept at 37° C. in the vortex shaker. The actual requirement for fibrinogen solution must be adjusted to the number of specimens. The solution is stable at room temperature for 48 h.

The glass plate is marked at a length of 16 cm and at the marking provided with a cross piece of foam plastic sealing tape (Tesa Moll item No. 05459-00047). Then a border of plastic sealing tape is drawn around the marked portion of the glass plate and glued to the cross piece in a liquid-tight manner. The wound dressing is punched out/cut out in a size of 160×50 mm. Then 150 mg of fibrinogen are dissolved in 60 ml of PBS buffer solution. The thrombin solution is produced by dissolving 10 mg of thrombin in 10 ml of BPS buffer solution and from this solution 1 ml is again diluted with 9 ml of BPS buffer solution. The fibrinogen solution and thrombin solution are kept at 37° C. For each prepared glass plate, 25 ml of fibrinogen solution and 2.5 ml of thrombin solution are required. For this purpose, both solutions are mixed and immediately combined for around 10 seconds in the vortex shaker and then poured on the glass plate and uniformly distributed. After this, the applied solution is dried for 72 h in a normal climate (23° C., 50% relative humidity). Before the separating strength measurement, the foam plastic border is removed from the glass plate.

The sample is punched out in a size of 100×40 mm. The test piece is rolled using a D 4271/1 Tape Applicator from the Sondes Place Research Institute Co., Surrey, England, with 20 N per cm in a defined manner and allowed to set for 20 minutes. The glass plate prepared in this way is placed in 900 traction device of the Z-005 traction and extension machine of the Zwick-Roell Co., Ulm, Germany and the free end of the reinforcement tape which extends over the specimen is inserted in the upper clamp. The force progression is measured with a constant traction rate of 100 mm/min. After detachment of the specimen, the same sample is again rolled after 20 minutes and again measured. In all, 4 measurements are made with the same specimen on the same fibrin layer. The force calculation is in accordance with DIN ISO 6133, method C.

8) Test of wound adhesion to the wound model agar/fibrin (per the Deutsche Apotheker Zeitung 131, Vol. No. 41, 2092-2094 (1991))—Test 8

In an in-vitro test, the adhesion tendency of the wound dressings with fibrin net formed on an agar surface is tested. The following reagents are required:

i) Agar-agar, item No. 1.0164, Merck Co.

ii) Human plasma from the DRK Blood Donation Service, apportioned and frozen at −18° C.

iii) Actin FS, item No. B4218-20 or B4218-100, DADE BEHRING Co., Eschborn Germany

iv) Calcium chloride solution 25 mm: 3.67 g calcium chloride dehydrate are dissolved with demineralized water to 1000 ml.

With the agar-agar and demineralized water, a 1.5% solution is produced by heating to the boiling point. After cooling to around 50° C., 10 ml of solution is pipetted into a Petri dish, covered, and allowed to cool. A portion of human plasma is thawed in a water bath at 37° C. and tempered.

(The following) are pipetted successively onto an agar plate:

1. 750 μl of calcium chloride solution, 25 mM

2. 1500 μl of human plasma

3. 750 μl of actin F

The substances are mixed well by swirling and thermostatted without a cover for 30 minutes in a conditioning cabinet at 37° C./50% relative humidity. In this time the fibrin net begins to form on the agar layer in a gel-like manner. The wound dressing to be tested is cut to a size of 2×2 cm. The cut sample is placed on the fluid, still not fully formed, fibrin net and pressed down lightly. Then the agar plate with the wound dressing (without cover) is placed for another 90 minutes in the conditioning cabinet. The wound dressing is now removed from the agar plate with pincers and visually or microscopically examined. The state of the fibrin net after removal of the wound dressing is evaluated. Here a wound dressing is termed as not wound-adherent, when a) the fibrin net is not damaged or b) the fibrin net shows few or minimal defects. Conversely, a wound dressing is classed as wound-adherent, when a) the fibrin net tears or is damaged when the wound is removed or b) remnants of the wound dressing are left behind the fibrin net.

### B) Composition of the Polyurethane Elastomer in Accordance with the Disclosure

TABLE 1
(data in % by weight)

| | Polyurethane | Polyurethane | Polyurethane | Polyurethane |
|---|---|---|---|---|
| Polyol Component (A) | Elastomer 1:A | Elastomer 2:A | Elastomer 3:A | Elastomer 4:A |
| Polyether polyol | About 99.4 | About 99.4 | About 99.4 | — |
| (Levagel VPKA 8732; | | | | |
| Bayer); OHZ 35 | | | | |
| Trifunctional poly- | — | — | — | About 99.5 |
| propylene ether polyol | | | | |
| (Desmophen 5034 | | | | |
| BT; Bayer) | | | | |
| GABCO 33LV (Air | — | — | — | About 0.5 |
| Products) | | | | |
| B(III) catalyst (here: | About 0.4 | About 0.4 | About 0.4 | — |
| neodecanoate; Coscat | | | | |
| 83; Cosan Chemical | | | | |
| Corp.) | | | | |
| Stabilizer (here: | About 0.2 | About 0.2 | About 0.2 | — |
| tocopherol) | | | | |
| Isocyanate | Mixture ratio | Mixture ratio | Mixture ratio | Mixture ratio |
| component (B) | A/B | A/B | A/B | A/B |
| Prepolymer on base | — | — | — | About |
| (HDI) (Desmodur | | | | 2.48:1.00 |
| E305; Bayer); NCO | | | | |
| content around 13% | | | | |
| Aliphatic prepolymer | About | About | About | — |
| on base (IPDI) | 2.70:1.00 | 3.45:1.00 | 2.34:1.00 | |
| (Desmodur VP LS | | | | |
| 2371); NCO content | | | | |
| around 3.7% | | | | |

The polyurethane elastomer is obtained in a short reaction time by adding both components at room temperature and mixing them homogenously. After a short time, during which the reaction between the two components starts, the viscosity grows steadily. The reaction can be completed by adding heat.

### C) Manufacture and Design of the Wound Dressings

### Exemplary Embodiment 1

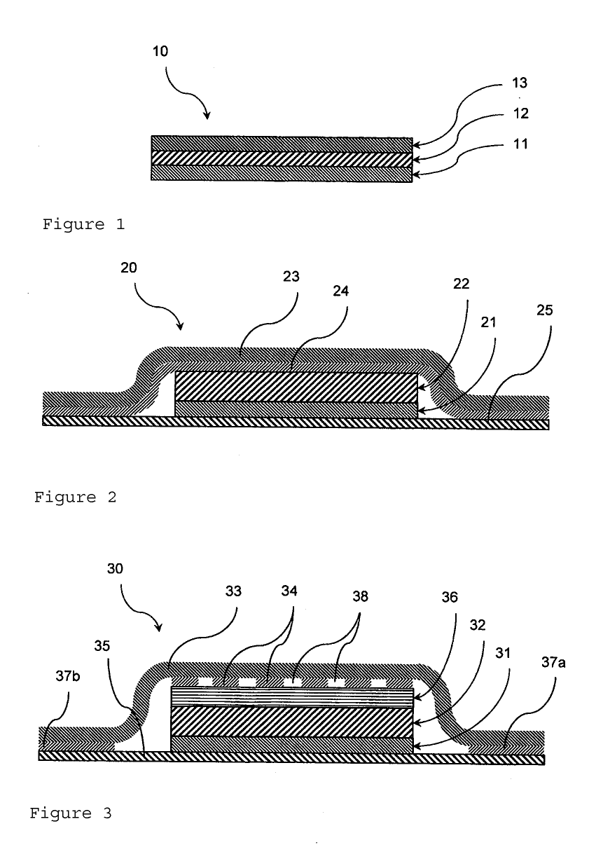

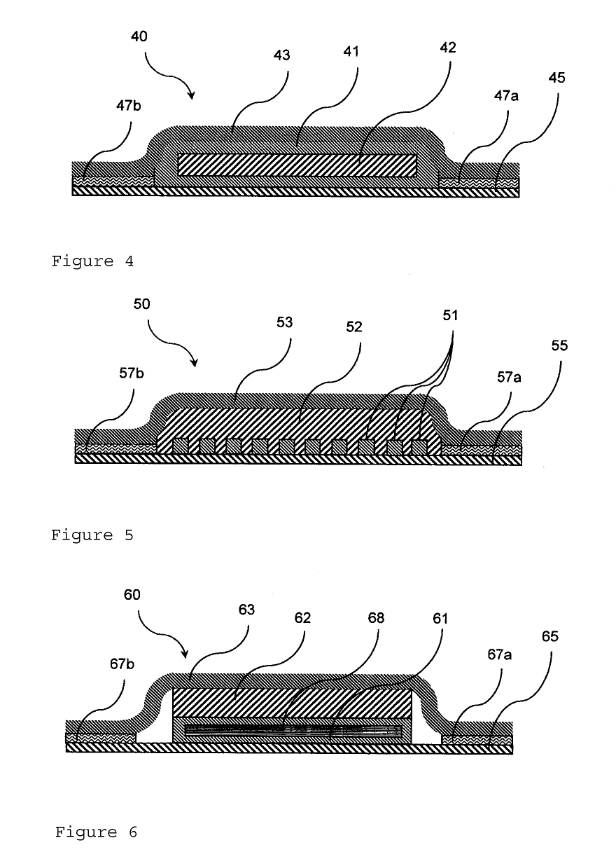

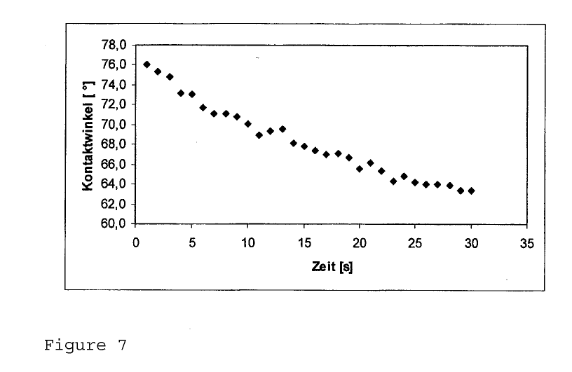

The adhesive polyurethane elastomer 1 (Collano Co., Sempach-Station, Switzerland) was used as the wound contact layer. This adhesive comprises of a polyether-polyol component (A) and a cycloaliphatic isocyanate component (B). Then 100 g of component A is mixed with 37 g of component B homogenously. This mixture is applied to silicon paper Separacon 9120-60 of the Maria Soell Co., Nidderau, Germany, with a layer thickness of around 200 μm over the entire surface. After a short time, during which the reaction between the two components starts, the viscosity increases steadily. Then PermaFoam (thickness around 5 mm, item No. 409-401) from the company Paul Hartmann AG, Heidenheim, Germany is added to this forming polyurethane layer. Subsequently, a wound dressing of 10×10 cm is punched out. Thus the design of this wound dressing corresponds to that shown schematically in FIG. 1.

TABLE 2
Properties of the exemplary embodiment 1:

| Test Method | Unit | Example 1 | PermaFoam |
|---|---|---|---|
| Free absorption (Test 3) | g/g | About 9.6 | About 10.1 |
| Water vapor permeability | g/m²/24 h | About 307 | About 301 |
| (Test 4a) | | | |
| Fluid uptake capacity (Test 5) | g/m²/24 h | About 6451 | About 6765 |
| Adhesive strength 90° (Test 1) | N/25 mm | About 0.09 | — |
| Contact angle (Test 6) | ° | — (1 | — |

(1 - not measurable, as sudden spread of the drop after a few seconds is observed.

Exemplary embodiment 1 shows that with an additional application of a hydrophilic wound contact layer, the performance properties of a wound dressing without a wound contact layer are not changed and/or not substantively changed. Here in particular the water vapor permeability and the maximal fluid uptake capacity are to be highlighted (cf. Table 2). The hydrophilic wound contact layer of example 1 possesses low adhesive strength on steel, whereby it was determined in a bearing test, that an adhesive capacity on dry skin is adequate for a first fixation of a wound dressing. Also, exemplary embodiment 1 showed in accordance with the test method “Test of wound adhesion on an agar/fibrin wound model” (Test 8) no damage to the fibrin layer after removal of the wound dressing. The wound dressing thus should not be classed as wound-adherent.

TABLE 3
Testing of repeated adhesion of exemplary embodiment 1 on the
same surface in accordance with test method Test 7:

| | | Maximal |
|---|---|---|
| | Average force | measured force/ |
| Test run/min | N/40 mm | N/40 mm |
| 1. Traction t = 0 | About 0.39 | About 0.56 |
| 2. Traction t + 20 | About 0.37 | About 0.53 |
| min | | |
| 3. Traction t + 40 | About 0.38 | About 0.53 |
| min | | |
| 4. Traction t + 60 | About 0.39 | About 0.56 |
| min | | |

Exemplary embodiment 1 showed in accordance with the test method of Test 7 that this wound dressing does not lose its adhesive strength even after repeated removal of the wound dressing.

### Exemplary Embodiment 2

### To Determine the Individual Properties of the Wound Contact Layer or the Hydrophilic Polyurethane Elastomer

The polyurethane elastomer 1 was placed on a polyurethane film (VP940-2) of the Collano-Xiro Co., Buxtehude, Germany with an application weight of 200 g/m². This laminate was produced, in order to determine the free absorption of the polyurethane elastomer as well as its contact angle. To determine the free absorption, the weight of the polyurethane film was subtracted. The free absorption was determined in accordance with Test 3. The tests were done no sooner than 3 days after manufacture of the laminate.

### Exemplary Embodiment 3

### To Determine the Individual Properties of the Wound Contact Layer or the Hydrophilic Polyurethane Elastomer

The polyurethane elastomer 2 was placed on a PU carrier film (polyurethane film VP940-2, Collano-Xiro Co., Buxtehude, Germany) with an application weight of 60 g/m2. This laminate was manufactured in order to determine the free absorption of the polyurethane elastomer as well as its contact angle. To determine the free absorption, the weight of the polyurethane film was subtracted. The free absorption was determined in accordance with Test 3. The tests were done no sooner than 3 days after manufacture of the laminate.

TABLE 4
Properties of exemplary embodiments 2 and 3

| | | | | | Adhesive |
|---|---|---|---|---|---|
| | Absorption | Contact | MVTR/ | MVTR (#/ | strength/ |
| | g/g | angle/° | g/m 2/24 h | g/m 224 h | N/25 mm |
| | (Test 3) | (Test 6) | (Test 4a) | (Test 4b) | (Test 2) |
| Example 2 | About 0.92 | — (1 | About 1331 | About 2361 | About 0.20 |
| Example 3 | About 0.60 | About 63 (2 | About | About 2745 | About 2.84 |
| PU carrier | 0 | — | About 2598 | About 4317 | — |
| film | | | | | |

(# With an application weight of 100 g/m²
(1 - not measurable, as sudden spreading of the drop was observed after a few seconds; the surface must therefore be termed as very hydrophilic.
(2 - Contact angle 30 seconds after application of the drop on the adhesive PU gel side of exemplary embodiment 3. The intense change in the contact angle depending on the time shows the hydrophilic nature of exemplary embodiment 3 (cf. FIG. 7).

These examples can be used to show that the utilized elastomers or the wound contact layers are hydrophilic and possess good free absorption as well as good water vapor permeability.

It should be noted that the disclosure is not limited to the embodiment described and illustrated as examples. A large variety of modifications have been described and more are part of the knowledge of the person skilled in the art. These and further modifications as well as any replacement by technical equivalents may be added to the description and figures, without leaving the scope of the protection of the disclosure and of the present patent application.

What is claimed is:

1. A multi-layer wound dressing comprising a carrier layer, an absorbent layer and a hydrophilic wound contact layer, wherein the wound contact layer is connected to the absorbent layer and comprises a polyurethane elastomer.

2. The wound dressing of claim 1, wherein the absorbent layer is integrally connected to the wound contact layer.

3. The wound dressing of claim 1, wherein the elastomer is an adhesive polyurethane elastomer or a polyurethane elastomer adhesive.

4. The wound dressing of claim 1, wherein the polyurethane elastomer is a polyurethane adhesive which comprises less than about 4% by weight water.

5. The wound dressing of claim 1, wherein the wound contact layer is a polyurethane elastomer which is obtainable by polymerization of at least one aliphatic or cycloaliphatic isocyanate component with a polyether polyol component.

6. The wound dressing of claim 1 wherein the wound contact layer is a polyurethane elastomer, preferably a polyurethane elastomer adhesive, wherein the elastomer is obtained by polymerization of isophorone diisocyanate or a modified isophorone diisocyanate with at least one diol or polyol component.

7. The wound dressing of claim 6 wherein the at least one diol or polyol component has 2, 3, 4, 5, or 6 hydroxyl groups.

8. The wound dressing of claim 6 wherein the at least one polyether polyol has OH numbers of about 20 to about 112.

9. The wound dressing of claim 6 wherein the at least one polyether polyol has an EO content of greater than about 10% by weight.

10. The wound dressing of claim 6 wherein the at least one polyether polyol has an EO content of about 10% to about 40% by weight.

11. The wound dressing of claim 6 wherein the at least one polyether polyol has an EO content of about 10% to about 20% by weight.

12. The wound dressing of claim 1 wherein the wound contact layer has an adhesive strength of about 0.02 to about 5 N/25 mm.

13. The wound dressing of claim 1, wherein the wound contact layer is a discontinuous wound contact layer.

14. The wound dressing of claim 1, wherein the absorbent layer a hydrophilic polymer film, an absorbent pad or nonwoven, a polymer matrix comprising at least one hydrocolloid, a freeze-dried foam, or combinations thereof.

15. The wound dressing of claim 1, wherein the absorbent layer comprises a hydrophilic polyurethane foam.

16. The wound dressing of claim 1 having a maximal fluid uptake capacity of at least about 5000 g/m²/24 h.

17. The wound dressing of claim 1, wherein the carrier layer comprises a polyurethane foam or polyurethane film.

18. The wound dressing of claim 1, wherein the carrier layer and the absorbent layer together comprise a laminate of two different polyurethane foams.

19. The wound dressing of claim 1, wherein the absorbent layer has a higher free absorption than the wound contact layer.

20. The wound dressing of claim 1, wherein the hydrophilic wound contact layer further comprises at least one antimicrobial agent.

21. The wound dressing of claim 1, wherein the wound dressing further comprises at least one distribution layer.

22. A method of treating a chronic wound comprising contacting the wound with a hydrophilic wound contact layer comprising a hydrophilic polyurethane elastomer.

23. The method of claim 22 wherein treating further comprises actively or passively supporting tissue buildup in the wound.
